(19) United States
(12) Patent Application Publication
(10) Pub. No.: US 2026/0220337 A1
(43) Pub. Date: Jul. 30, 2026

(54) SYSTEMS AND METHODS FOR QUANTITATIVE ANGIOGRAPHY USING PHYSICS-INFORMED NEURAL NETWORKS

(71) Applicant: The Research Foundation for The State University of New York, Amherst, NY (US)

(72) Inventors: Ciprian IONITA, Buffalo, NY (US); Kyle WILLIAMS, Amherst, NY (US); Mohammad Mahdi Shiraz BHURWANI, Buffalo, NY (US)

(21) Appl. No.: 19/158,219

(22) PCT Filed: Feb. 21, 2024
(86) PCT No.: PCT/US2024/016787

§ 371 Date: Aug. 20, 2025

Related U.S. Application Data
(60) Provisional application No. 63/486,248, filed on Feb. 21, 2023.

(51) Int. Cl. G06F 30/27; G06F 30/28

(52) U.S. Cl. CPC G06F 30/27; G06F 30/28

(57) ABSTRACT

A computer-implemented methods and systems are provided for predicting flow characteristics in a vasculature. The method includes obtaining time-series angiography data of a vasculature; determining vessel wall geometry within the vasculature; generating, using one or more processors, a set of collocation points within the time-series angiography data co-registered with the vessel wall geometry; and determining, using the one or more processors, a flow velocity and pressure at each collocation point using a physics-informed neural network (PINN) having an objective function based on the Navier-Stokes equation, the convection equation, and boundary conditions including the vessel wall geometry and the angiography data. In some embodiments, determining the flow velocity and pressure at each collocation point comprises minimizing a difference between a contrast intensity measured at each collocation point of the angiography data and an imputed contrast intensity calculated from the PINN-determined flow velocity and pressure at each collocation point.

### CROSS-REFERENCE TO RELATED APPLICATIONS

This application claims priority to U.S. Provisional Application No. 63/486,248, filed on Feb. 21, 2023, now pending, the disclosure of which is incorporated herein by reference.

### STATEMENT REGARDING FEDERALLY SPONSORED RESEARCH

This invention was made with government support under contract no. R01 EB030092 awarded by the National Institutes of Health (NIH). The government has certain rights in the invention.

### FIELD OF THE DISCLOSURE

The present disclosure relates to angiographic analysis, and in particular, using artificial intelligence to analyze angiographic imagery.

### BACKGROUND OF THE DISCLOSURE

Computational fluid dynamics (CFD) has long been the gold standard in iterative hemodynamic calculations, especially with respect to resolution of velocity and pressure fields. However, for correct algorithmic convergence to occur, CFD requires careful selection of initial and boundary conditions, many of which rely on assumptions or approximations from literature values. This is not particularly troublesome for many applications of CFD, where input velocities and pressures for the system are known. However, for medical applications such as, for example, patient-specific vessel models derived from CT angiography (CTA), these input functions are not inherently known. In the research setting, such conditions may be approximated from literature to yield approximated velocity and pressure distributions throughout the vessel of interest. However, the patient specificity of flow (outside the solution space geometry) is lost in the solution.

Additionally, the process of generating CFD results from patient-specific vasculature models is computationally expensive and time intensive. Once a volume rendering of a vessel is performed using CTA, the model is imported into a meshing software to manually remove artifacts or undesired structures as well as identify regions of the model (inlets, outlets, vessel walls and vessel lumen). Once the mesh is generated, it serves as an input to a CFD software such as ANSYS, where, based upon a user-defined inlet velocity function, no slip condition at the walls, and zero pressure at the model outlets, an iterative solver generates velocity and pressure fields by solving the Navier-Stokes equation for incompressible fluid flow. This finite element analytical method often requires several hours for stable convergence of velocity and pressure distributions, provided the method reaches convergence based upon the input arguments. Should the model fail to converge, the solution must be re-run under different conditions (most often by varying the mesh density or temporal resolution of the solver).

With the time, manual manipulation, and computational expense of the CFD velocimetry method, clinical implementation of the solver is difficult, and rapid feedback is generally not feasible. This aside, the method produces results which conform to the Navier-Stokes model of fluid flow given the tortuous geometries present in patient-specific neurovasculature, yielding valuable comparative data for simpler, more efficient velocimetry algorithms. As a result, CFD velocimetry results are often used as a benchmarking tool for imaging-based techniques, such as gradient-based methods and particle tracking techniques. Although each of these image-based methods has shown good agreement with CFD velocimetry results, indicating some level of agreement with the Navier-Stokes equation, gradient-based methods suffer from artifacts due to quantum mottle in the image, and radiopaque particles cannot yet be used for in vivo imaging.

One of the main challenges of CFD is the high computational cost and time required to solve the PDEs on a discretized domain. Depending on the complexity of the geometry, boundary conditions, and fluid properties, CFD simulations can take hours or even days to run on high-performance computers. This makes CFD impractical for clinical applications, such as intraoperative guidance or emergency decision-making. Moreover, CFD simulations are sensitive to the choice of numerical schemes, mesh resolution, and turbulence models, which can introduce errors and uncertainties in the results. Furthermore, CFD simulations often rely on assumptions or estimations of the boundary conditions, such as the inlet and outlet flow rates and pressures, which may not reflect the actual physiological state of the patient. These factors limit the accuracy and reliability of CFD for hemodynamic assessments in the neurovascular field.

There is a need for techniques for accurate hemodynamic determination in less time than CFD and with the ability to derive all initial and boundary conditions needed for convergence from imaging data.

### BRIEF SUMMARY OF THE DISCLOSURE

The present disclosure provides the use of physics-informed neural networks (PINNs), a class of neural networks within the fully connected deep neural network (DNN) family. The medical imaging-assisted PINN is capable of resolving patient-specific vascular flow fields with no user input, and in a manner which respects the Navier-Stokes and convection governing equations, as well as the network's adherence to the imaging data itself. Embodiments provide an important step toward personalized medicine in the neurovascular space. This present disclosure demonstrates the feasibility of data-driven neurovascular informatics based upon neural networks which are forced to respect governing physical equations.

Diagnostic procedures for neurovascular diseases often employ angiography, where Quantitative Angiography (QA) is specifically utilized to evaluate contrast flow. Despite the effectiveness of traditional angiography, it presents an ill-posed problem when extracting local dynamic information from angiogram images. Embodiments of the present disclosure provide techniques using Quantitative Angiography (QA) data as a novel form of constraint via the convection equations for iodinated contrast agents, thereby obviating the need for exhaustive knowledge of boundary conditions. This innovative strategy improves the accuracy and robustness of diagnostic methods in neurovascular health and establish a practical workflow for utilizing PINNs to solve the Navier-Stokes equations specific to neurovascular diseases.

Use of the present techniques of determining flow characteristics within a vasculature for patient assessment. For example, the present techniques may be used to predict six-month treatment outcomes, with an 80% accuracy. In another example, the present techniques may be used to predict the likelihood of intracranial aneurysm (IA) occlusion using pre- and post-treatment 2D-QA maps.

The presently-disclosed use of PINNs present a transformative potential for peri-procedural hemodynamic assessments, obviating the need for direct correlations between imaging biomarkers and hemodynamic states. PINNs adeptly integrate physical principles within a neural network framework, facilitating the resolution of complex partial differential equations (PDEs) without the constraints of traditional numerical discretization or comprehensive boundary conditions. Moreover, PINNs can assimilate data from diverse measurements, offering patient-specific insights that are more precise and robust than those derived from conventional Computational Fluid Dynamics (CFD). While the efficacy of PINNs in solving fluid dynamic PDEs is well-documented, their application within clinical environments is nascent.

### DESCRIPTION OF THE DRAWINGS

For a fuller understanding of the nature and objects of the disclosure, reference should be made to the following detailed description taken in conjunction with the accompanying drawings, in which:

FIG. 1: Digitally-subtracted 1000 frame per second (fps) angiographic acquisition (A), its corresponding binary mask used to define the system geometry (B), and the Sobel-filtered binary mask, used to define the walls of the model (C).

FIG. 2: Collocation points (A) defined in black, with inlet and outlet boundaries overlaid as labeled. The complete domain (B) shows collocation points (black), vessel walls (dark gray), the inlet (light gray, labeled) and outlets (light gray, labeled) of the vessel at a single time point.

FIG. 3: X and Y component of the velocity (u,v), pressure gradient is shown in the right snapshot.

FIG. 4: Progression from time series of 2D angiographic images to a boundary condition map to time-resolved collocation points to time-resolved contrast intensities.

FIG. 5: Results of a test embodiment using in vivo HSA of a carotid bifurcation model.

FIG. 6: Results of another test embodiment using in silico HSA of an aneurysm model.

FIG. 7: Results of another test embodiment using in silico HSA.

FIG. 8: Examples of contrast prediction from a PINN according to the present disclosure (left) compared to HSA image data (right).

FIG. 9: Time-resolved (top) and temporally-averaged (bottom-left) collocation point contrast intensities correspond well with real HSA data (bottom-right).

FIG. 10: Progressive angiographic frames obtained during neurointerventional procedures are shown from two perspectives (A: View 1, B: View 2), which exemplify the type of datasets available to clinicians. Angiography enables quantitative analysis of vascular morphology—particularly vessel calibers and aneurysmal dimensions—and provide a qualitative assessment of hemodynamic changes throughout the procedure. The rightmost panels depict a quantitative angiographic (QA) analysis utilizing peak contrast opacification within the sequence. The images incorporate computational fluid dynamics (CFD) simulations to enhance the visualization of hemodynamic behavior and support the discussion in the corresponding section, data is generated every 33 milliseconds to simulate a 30 frames pers second acquisition.

FIG. 11: Angiogram runs are used to extract the flow domain and the wall. An algorithm is used to create the flow domain for every frame. Z-direction in geometry definition represents the frame time. The algorithm also extracts inlet, outlet and walls sampling domains. Note, that in order for the PINNS to converge each structure is actually replaced by a cloud of points. Space and time location are inserted in the neural network and the loss function is defined using Navier Stokes equations, convection equation, zero wall velocity and zero outlet pressure. The solution after 10K iteration is shown in the output. Execution time is strongly dependent on the number of point in the space and time domain. For practical considerations we will focus only on the lesion of interest.

FIG. 12: Demonstration of the potential errors for contrast distribution assessment This figure presents an angiogram of a vascular structure, observed from two distinct perspectives, termed VIEW 1 and VIEW 2. The graph to the left illustrates variations in the arterial input function (AIF), comparing two measurements AIF1 and AIF2 derived from the respective ROIs at the inlet, emphasizing the impact of ROI placement on contrast assessment. Disparities in the AIF intensity between the two views are attributed to alterations in the X-ray path length through the vascular geometry. The blue dotted squares highlight the aneurysm at two different projections, not the clear difference in pattern and intensity even though this is the same angiogram. The plot on the right displays a line profile of the contrast intensity across the vessel lumen at the inlet, where the shaded region indicates a notable decrease in intensity. This drop-off suggests a misleadingly steep contrast gradient near the vessel walls when viewed from a projection not accounting for the cylindrical vessel geometry. Such an oversight can potentially skew the accuracy of contrast transport equation outcomes by falsely indicating a gradient where the actual distribution is more uniform.

FIG. 13: Deep learning reconstruction of bolus transition from a virtual angiogram using only 22 projections. A 3D virtual angiogram was used to create a C-arm rotational DSA acquisition with a two-second-long simulated bolus injection. Projections of the virtual angiograms are shown in the left column. Ground-truth, truncated, and data-driven reconstructions are shown in the middle. A comparison of the 3D volumes for ground truth and data-drive reconstruction is shown in the right column. DICE coefficient=0.94.

FIG. 14: Reconstruction of bolus arrival in a carotid, clinical data. (real angio data).

### DETAILED DESCRIPTION OF THE DISCLOSURE

Physics-informed neural networks (PINNs) are powerful tools, increasing in popularity for their ability to iteratively resolve fluid flow conditions that adhere to partial differential equations (PDEs) defined in fluid mechanics literature. Individual higher-order derivative terms included within PDEs are resolved by the inherent differentiation of terms within neural networks, allowing definitions of all terms within desired PDEs. Algebraic manipulation of PDEs to set one side of the equation equivalent to zero allows the conversion of the PDE to a loss term, which may be minimized by the network, ensuring the PDE of interest is considered when predicting flow conditions. In embodiments of the present disclosure, additional constraints, such as initial conditions (ICs) and boundary conditions (BCs), are also considered by minimizing the sum of the square differences (SSD) between network-predicted ICs and BCs and user-defined ICs and BCs. Together, these considerations can be combined into a single loss function, which, when solved iteratively, constrains the output flow characteristics to both the PDE of interest, and to the ICs and BCs of the system.

Within the neurovascular space, knowledge of blood velocity fields and blood pressure is often advantageous to assess risk factors associated with neurovascular diseases, such as aneurysm rupture or atherosclerosis-induced ischemia. For this reason, a solution to the Navier-Stokes equation, a PDE relating velocity to pressure in incompressible fluids, provides significant insight into the flow conditions and risks associated with neurovascular pathologies. For example, velocity in the x-direction may be determined according to:

$$ρ ( {u}_{t} + {uu}_{x} + {vu}_{y} ) = - {p}_{x} + μ ( {u}_{xx} + {u}_{yy} ) , \qquad ( 1 )$$

Where x, y, t are spatial/temporal coordinates determined from a series of angiograms showing flow of a contrast agent, p is pressure, u is flow velocity in the x direction, v is flow velocity in the y direction, and ρ (blood density) and (blood viscosity) are determined from biological averages. uₜ, uₓ, u_{y}, pₓ, uₓₓ, and u_{yy} are derived parameters which may be calculated using gradient descent, for example, tensorflow gradients (e.g, uₜ=tf.gradients(u,t)).

PINNs may be used to solve Navier-Stokes, provided ICs, BCs, and the solution geometry are properly defined. For example, a PINN may be used to iteratively solve the Navier-Stokes and convection equations for blood flow while minimizing assumptions and computational expense. In other words, partial derivatives of contrast density, x-velocity, y-velocity, and pressure (C, u, v, p) are calculated with respect to spatial and temporal coordinates (x, y, t). The inputs to the network will include (x, y, t) coordinates, and the network will predict (C, u, v, p) such that (as a function of the neural network (NN)):

$$\{ C , u , v , p \} = N N ( \{ x , y , t \} ) \qquad ( 2 )$$

Real HSA contrast media propagation from a time series of angiograms during contrast injection can be used as a boundary condition. In some embodiments, common assumptions may be used, such as, for example, a no slip condition at the walls of the vessel geometry.

As such, in some embodiments, the components of the loss function are (other embodiments may include additional components or a subset of these components):

### 1. Navier-Stokes Equation (2D)

$$ρ ( \frac{\partial u}{\partial t} + u \frac{\partial u}{\partial x} + v \frac{\partial u}{\partial y} ) + \frac{\partial p}{\partial x} - μ ( \frac{{\partial }^{2} u}{\partial {x}^{2}} + \frac{{\partial }^{2} u}{\partial {y}^{2}} ) = {ε}_{1} \qquad ( 3 )$$

$$ρ ( \frac{\partial v}{\partial t} + u \frac{\partial v}{\partial x} + v \frac{\partial v}{\partial y} ) + \frac{\partial p}{\partial y} - μ ( \frac{{\partial }^{2} v}{\partial {x}^{2}} + \frac{{\partial }^{2} v}{\partial {y}^{2}} ) = {ε}_{2} \qquad ( 4 )$$

### 2. Convection Equation (2D)

$$\frac{\partial C}{\partial t} + u \frac{\partial C}{\partial x} + v \frac{\partial C}{\partial y} + Pec ( \frac{{\partial }^{2} C}{\partial {x}^{2}} + \frac{{\partial }^{2} C}{\partial {y}^{2}} ) = {ε}_{3} \qquad ( 5 )$$

### 3. Continuity Equation (2D)

$$\frac{\partial u}{\partial x} = - \frac{\partial v}{\partial y} \to \frac{\partial u}{\partial x} + \frac{\partial v}{\partial y} = {ε}_{4} \qquad ( 6 )$$

### 4. Wall Loss (No-Slip Boundary Condition)

$$u ( WALL ) = v ( WALL ) = 0 mm / s \to u ( WALL ) = {ε}_{5} , v ( WALL ) = {ε}_{6} \qquad ( 7 )$$

### 5. Image-Based Loss (Contrast Pattern Match Boundary Condition)

$${C ( x , y , t )}_{p r e d} = {C ( x , y , t )}_{real} \to abs ( {C ( x , y , t )}_{p r e d} - {C ( x , y , t )}_{r e a l} ) = {ε}_{7} \qquad ( 8 )$$

Components 1, 2, and 3 represent governing physical equations, and components 4 and 5 represent boundary conditions. The network may be configured to respect each governing physical equation as well as all boundary conditions. In some embodiments, this is accomplished by taking the sum of the squares of each loss component (ε₁):

$$Loss = {( Navier Stokes Loss )}^{2} + {( Convective Loss )}^{2} + {( Continuity Loss )}^{2} + {( Wall Loss )}^{2} + {( Image Loss )}^{2} \qquad ( 9 )$$

$$or$$

$$Loss = {ε}_{1}^{2} + {ε}_{2}^{2} + {ε}_{3}^{2} + {ε}_{4}^{2} + {ε}_{5}^{2} + {ε}_{6}^{2} + {ε}_{7}^{2} \qquad ( 10 )$$

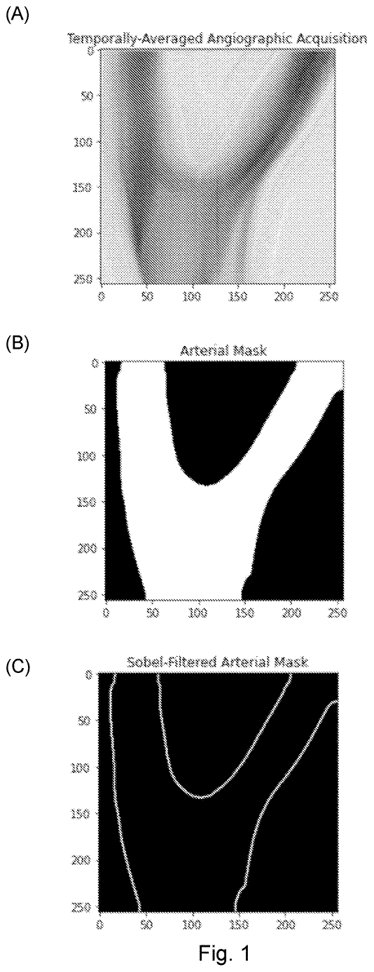

The physical domain may be extracted from in vitro acquisition of 2D angiography. In some embodiments, the acquisition is temporally averaged (FIG. 1A), and a threshold is used to mask the vessel of interest, shown in FIG. 1B. The coordinates of the vessel walls of the model (within the image sequence) may then be defined by passing a Sobel (high-pass) filter over the vessel mask, shown in FIG. 1C.

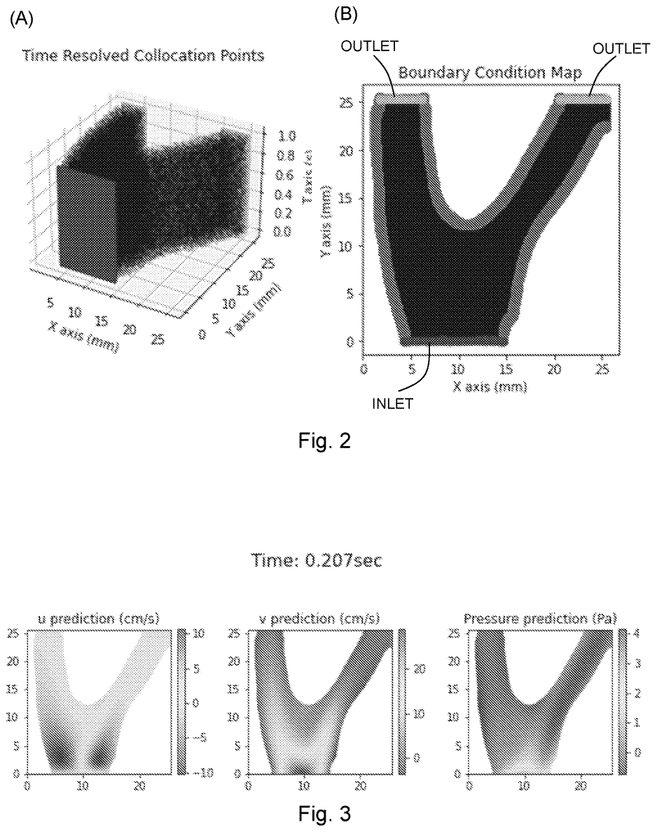

The collocation points (coordinates within the vessel for which the network makes predictions for velocity and pressure measurements) may be obtained by identifying the regions of the mask falling between vessel walls. For example, the x-coordinate bounds (in pixels) of the vessel at each row of pixels in the vessel mask are identified and concatenated to a list of coordinate bounds in the form [[x_min, y, t_min], [x_max, y, t_max]]. Since the bounds are defined at each row of the vessel mask, the y-coordinate remains constant between bounds, and may simply be denoted y to emphasize this equality. In regions with multiple vessels per given row (such as in the bifurcating region of the carotid bifurcation shown in the figures), the bounds of the vessel are reported separately for each segment. Iterating over this list of vessel boundaries, collocation points may be generated using, for example, Latin hypercube sampling (LHS), which uses upper and lower limits of a coordinate space, as well as an input number of points, to randomly sample the region of interest. In some embodiments, the solution space is randomly sampled in the spatial domain, and discretely sampled (i.e., at every time point) in the time domain. The number of points generated per row of the vessel geometry are width-dependent (allowing even sampling of the entire geometry), and may be be adjusted multiplicatively by a user-defined sampling density factor. Once defined, the collocation points can be rescaled to meaningful units leveraging the known pixel pitch of the detector used to generate the angiographic sequence. Similarly, points in the time dimension may be determined using the frame rate and number of frames. The collocation point matrix is shown in FIG. 2A. The inlet and outlets of the model are defined as the lower and upper limits of the geometry with respect to the y-coordinates. The fully resolved geometry, including the wall definitions, inlets and outlets, are observed in FIG. 2B.

The IC of this system may assume universally zero velocity and pressure (ensuring no assumptions of previous flow are predicted by the network). The BCs of the system are defined at the walls, the inlet(s) to the vessel of interest, and the outlet(s) out of the vessel of interest. The walls may be given a non-slip condition (i.e., velocity should be zero in both x- and y-directions), the outlets may be constrained to a zero-pressure condition (to give the network a direction of flow), and the velocity at the inlet may be defined by the user as any arbitrary function. It should be noted that the inlet and outlet constraints are optional, but may improve network convergence if used. In embodiments where ICs are enforced, IC constraints may only be enforced at t=0, not at collocation points.

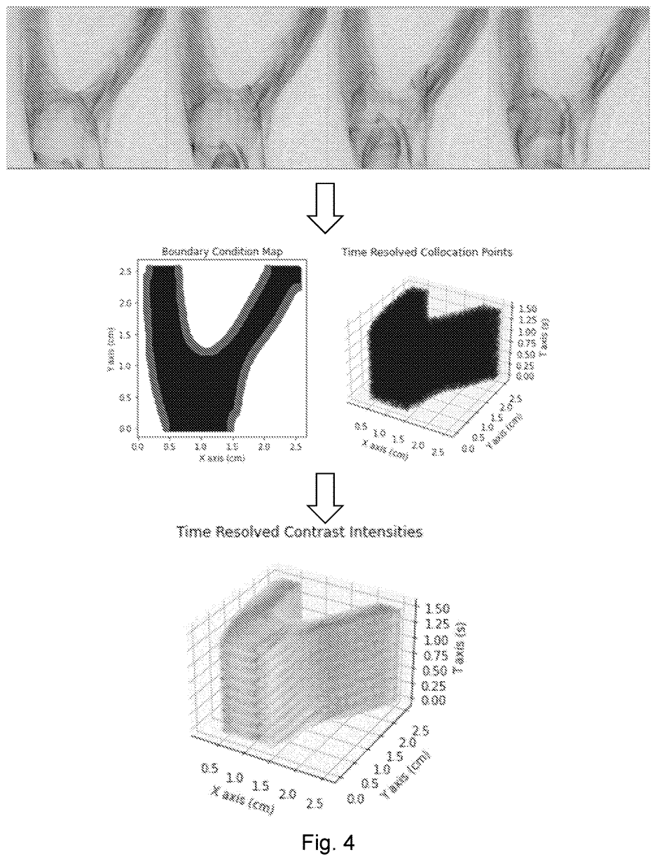

Using contrast information, a second equation can be added in the loss function, removing the need for initial flow boundary conditions. In addition to automatically defined luminal and vessel wall collocation point coordinates (x, y, t), another input, C, may be added for contrast intensity (resulting in time-resolved contrast intensities as shown in FIG. 4). This may be performed using, for example, nearest neighbor intensity assignment, such that:

$${C}_{collo} ( x , y , t ) = {C}_{HSA} ( int ( x ) , int ( y ) , int ( t ) ) \qquad ( 11 )$$

In some embodiments, the angiograms are co-registered with a 3D geometry of the vasculature. In embodiments using 2D angiograms, the contrast data from the image data can be adjusted using 3D vessel geometry(ies) to correct for foreshortening. In other words, the contrast density shown in 2D images of a vessel will vary across the 2D width of the vessel at a particular location along its centerline due to the cross-sectional geometry (3D) of the vessel at that location. By co-registering the 2D geometry of the vessel with its 3D geometry, the contrast density can be adjusted in the 2D images to remove the effect of the vessel thickness.

Using these conditions, network training, which typically ranges from 2-10 minutes under constant flow conditions (but could be more or less than this range), results in convergent, time-resolved results, with arbitrarily high, user-defined spatial and temporal resolution. Training time and convergence are highly dependent on spatial and temporal resolutions, as well as transience in the inlet velocity function, however, the method may be used to obtain computational fluid dynamics (CFD)-comparable results at a significantly more efficient rate.

In some embodiments, the equations of the loss function may be weighted differently from each other. In some embodiments, the loss components may be weighted equally. For example, the loss components may be combined into one loss value using the sum of the squares of each loss component. In some embodiments, one or more of the loss components may be assigned a different weight from one or more of the other loss components when training the network.

Once the physical domain is defined the velocity calculations are performed. Results of the PINNs are shown in FIGS. 3A-3C. FIGS. 3A and 3B shows the predicted x and y components of velocity (u, v), and FIG. 3C shows the predicted pressure gradient.

In an example embodiment, a PINN was used and structured as having three inputs (x,y,t), and four outputs (C, u, v, p), with 10 hidden layers and 250 nodes per layer. The network was trained for 750 epochs, and all collocations points were entered at each epoch. The total training time was 23 minutes.

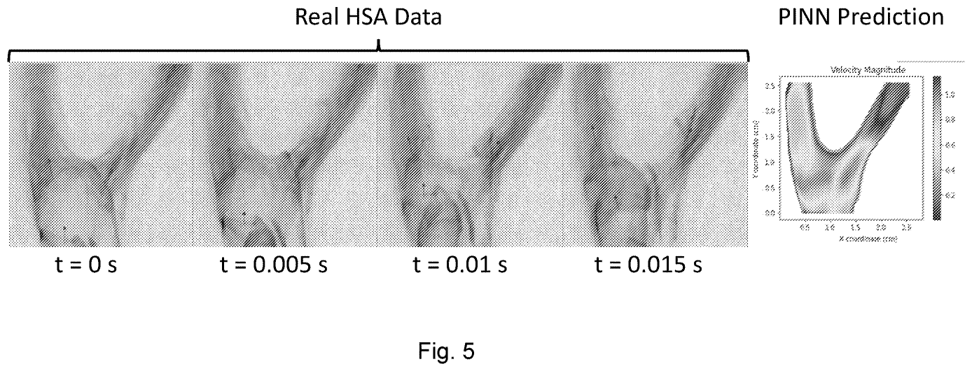

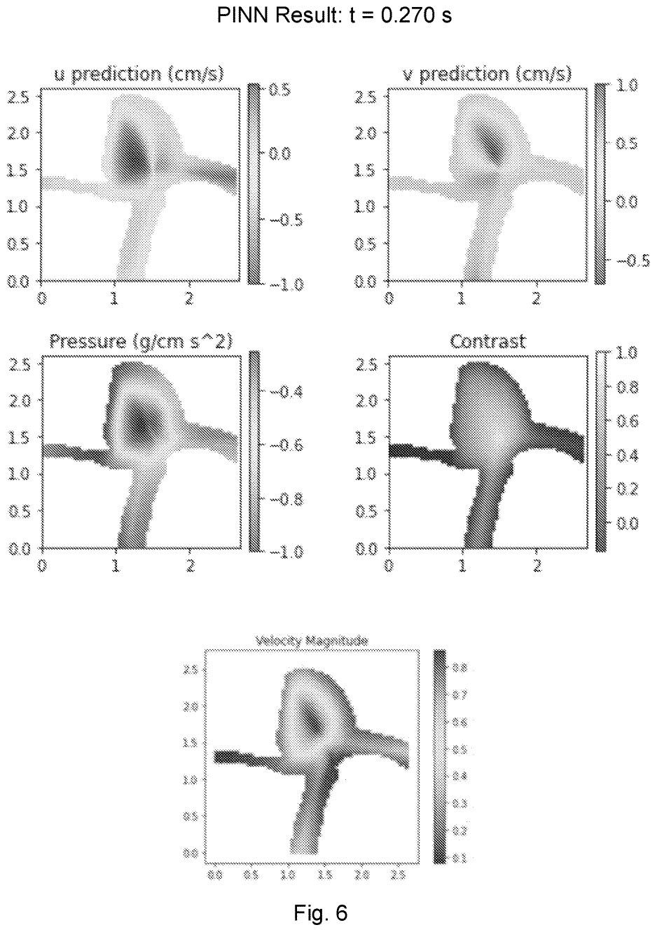

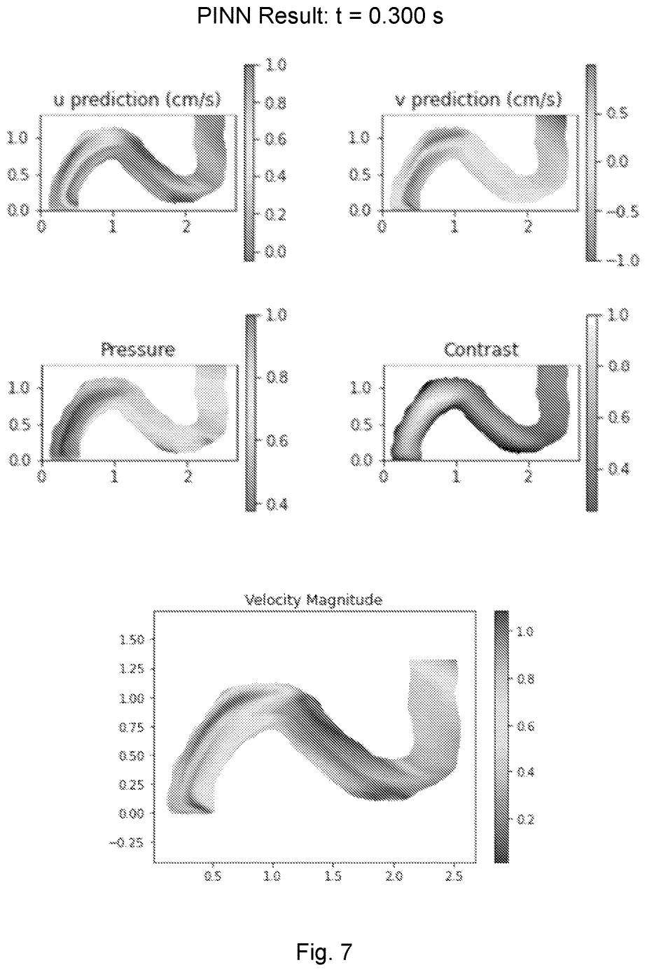

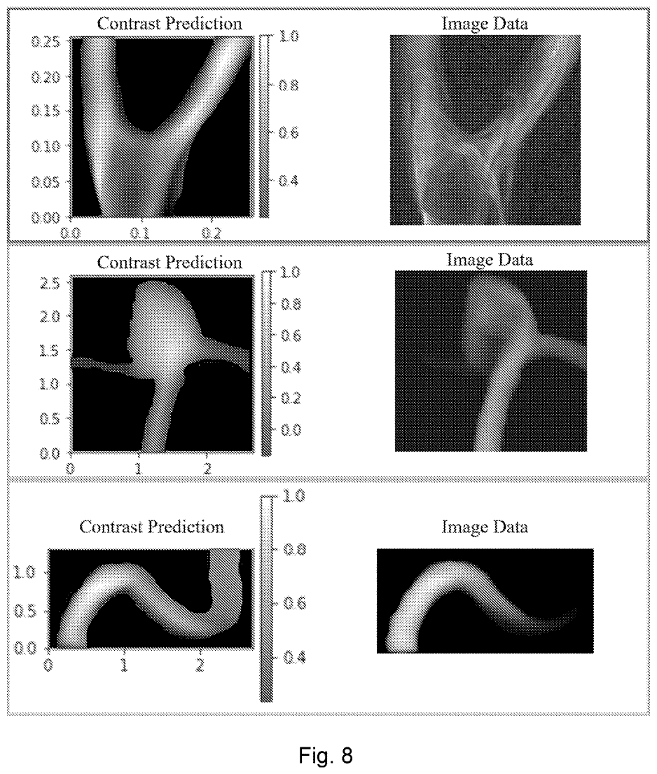

FIG. 5 shows a test embodiment using high-speed angiography (HSA) data obtained in vivo. Velocity predictions correlated well with contrast flow patterns through a test embodiment of a carotid bifurcation model. Velocity results normalized to the maximum for distribution clarity. FIG. 6 shows a test embodiment using HSA data obtained in silico—a vorticial flow pattern through an aneurysm model is shown at one time step. The low pressure zone in the middle of the vortex is also captured by the PINN output. FIG. 7 is an example demonstrating the convection equation—it can be seen that velocity and contrast propagation are closely correlated. FIG. 8 shows several examples of contrast prediction from a PINN according to the present disclosure compared to HSA image data, and showing good agreement between the predicted propagation and the actual images. It is noted that contrast predictions were not made outside the vessel boundaries, therefore background has been converted to black for ease of comparison.

The results indicate that contrast propagation from imaging data can be used as a velocity predictor, meaning manually defined inlet velocity functions are not necessary. The accuracy of the distributions are largely dependent on network adherence to contrast media propagation. PINNs represent a useful method of deriving patient-specific hemodynamic information from HSA imaging while preserving the fidelity of the data. Moreover, these results can be generated orders of magnitude faster than CFD simulation runtimes and do not require a priori or assumed knowledge of flow conditions.

With reference to FIG. 15, the present disclosure may embodied as a computer-implemented method 100 for predicting flow characteristics in a vasculature (e.g., a structure of one or more blood vessels). The method 100 includes obtaining 103 time-series angiography data of the vasculature. For example, the angiography data may be a series of 2D angiographic images taken during injection of a contrast agent. In other examples, the angiography data may be 3D data. Although the present disclosure is presented with reference to embodiments using x-ray data, other modalities may be used including combinations of modalities (e.g., magnetic resonance imaging (MRI), computed tomography (CT), etc.)

The vessel wall geometry of the vasculature is determined 106. For example, the 3D geometry of the vessel walls may be determined. The geometry may be determined by analyzing the angiography data, by analyzing other imaging data, or other techniques. In some embodiments, the vessel wall geometry is determined by receiving vessel wall geometry data. In some embodiments, the vessel wall geometry is determined from a model of the vasculature (e.g., a virtual 3D model, etc.) In an example, a 2D vessel wall geometry may be determined from a time series of 2D angiograms of the vasculature during contrast injection. For example, the 2D geometry may be extracted by averaging two or more of the time series of 2D angiograms to obtain a temporally averaged angiogram; thresholding the temporally averaged angiogram to determine a vessel mask; and filtering the vessel mask with a Sobel filter to obtain a 2D geometry of the vasculature. These are examples, and other techniques may be used.

A set of collocation points is generated 109 within the time-series angiography data co-registered with the vessel wall geometry, using one or more processors. The collocation points are determined in three dimensions (x,y,t) or four dimensions (x, y, z, t) as appropriate for the angiography data and vessel wall geometry. For example, in an embodiment where the angiography data is a time series of 2D angiograms, the collocation points may be determined by: determining minimum and maximum ranges for each of x, y, and t to define a solution space; and sampling the solution to generate a set of random collocation points (e.g., using a technique for random sampling such as, for example, Latin hypercube sampling, Monte Carlo sampling, etc.) In some embodiments, a 3D geometry of the vasculature may be co-registered with the 2D geometry, and contrast data may be adjusted at each collocation point based on the co-registered geometries to correct for foreshortening.

Flow characteristics (e.g., flow velocity, pressure, contrast intensity) at each collocation point are determined 112 using a physics-informed neural network (PINN) (using one or more processors). For example, in some embodiments, the method includes determining the flow velocity (in two or three dimensions) and the pressure. The PINN has an objective function based on the Navier-Stokes equation, the convection equation, and boundary conditions including the vessel wall geometry and the angiography data. The objective function may include the continuity equation. In some embodiments, the PINN has three inputs (collocation points in x, y, and t). In some embodiments, the PINN has four inputs (collocation points in x, y, z, and t). In some embodiments, the PINN has four outputs (C, u, v, and p). In some embodiments, the PINN has five outputs (C, u, v, w, and p—where u, v, w correspond to flow velocity in 3D). In some embodiments, the PINN is a fully connected neural network such that all inputs and all outputs are connected at each layer of the PINN. The PINN is configured to derive boundary conditions from the vessel wall geometry and the angiography data.

Determining 112 the flow velocity and pressure at each collocation point may include minimizing 115 a difference between a contrast intensity, Cᵣₑₐₗ, measured at each collocation point of the angiography data and an imputed contrast intensity, C_{pred}, calculated from the PINN-determined flow velocity and pressure at each collocation point. Determining the flow velocity and pressure may include generating an angiogram based on the flow velocity and pressure at each collocation point. For example, a 2D angiogram may be generated to aid in controlling convergence of the PINN. In another example, a 3D angiogram is generated to minimize an error in the determined flow velocity and pressure.

In some embodiments, additional loss function configurations may include known inlet velocity waveforms acquired via Doppler ultrasound and/or inlet pressure waves measured by pressure sensors. For example, the PINN boundary conditions may further include:

$$u ( {x}_{inl} , t ) = {u}_{int} ( t ) and p ( {x}_{inl} , t ) = {p}_{inl} ( t )$$

where uᵢₙₗ(t) and pᵢₙₗ(t) represent the inlet velocity and the pressure waveform. This scenario is not clinically irrelevant since in practice we could acquire Doppler waveform on the patient carotid and pressure could be estimated using pressure sensors connected to the guiding catheters.

The method may include determining, using the one or more processors, a state of occlusion of one or more vessels of the vasculature. Other states of disease (e.g., neurovascular diseases) may be determined—for example, aneurysm rupture, the efficacy of flow diverters in aneurysm healing, and the severity of intracranial arterial disease (ICAD) in stroke patients using patient-specific vascular phantoms.

In another aspect, the present disclosure may be embodied as a system for predicting flow characteristics in a vasculature. The system includes a processor, wherein the processor is configured to perform any of the methods disclosed herein.

For example, the processor may be configured to obtain time-series angiography data of a vasculature. The processor may determine a vessel wall geometry of the vasculature and generate a set of collocation points in based on the angiography data co-registered with the vessel wall geometry. The processor has a PINN configured to determine flow characteristics (one or more of velocity, pressure, and contrast intensity). The PINN may be configured with an objective function based on the Navier-Stokes equation, the convection equation, and boundary conditions including the vessel wall geometry and the angiography data. The objective function may include the continuity equation. In some embodiments, the PINN has three inputs (collocation points in x, y, and t). In some embodiments, the PINN has four inputs (collocation points in x, y, z, and t). In some embodiments, the PINN has four outputs (C, u, v, and p). In some embodiments, the PINN has five outputs (C, u, v, w, and p—where u, v, w correspond to flow velocity in 3D). In some embodiments, the PINN is a fully connected neural network such that all inputs and all outputs are connected at each layer of the PINN. The PINN is configured to derive boundary conditions from the vessel wall geometry and the angiography data.

The processor may be configured to determine the flow velocity and pressure at each collocation point by minimizing a difference between a contrast intensity, Cᵣₑₐₗ, measured at each collocation point of the angiography data and an imputed contrast intensity, C_{pred}, calculated from the PINN-determined flow velocity and pressure at each collocation point. Determining the flow velocity and pressure may include generating an angiogram based on the flow velocity and pressure at each collocation point. For example, a 2D angiogram may be generated to aid in controlling convergence of the PINN. In another example, a 3D angiogram is generated to minimize an error in the determined flow velocity and pressure.

In another example, the processor may be configured to extract a 2D geometry from a time series of 2D angiograms of the vasculature during contrast injection. For example, the 2D geometry may be extracted by averaging two or more of the time series of 2D angiograms to obtain a temporally averaged angiogram; thresholding the temporally averaged angiogram to determine a vessel mask; and filtering the vessel mask with a Sobel filter to obtain a 2D geometry of the vasculature.

The processor may be further configured to determine collocation points (x,y,t) of the time series of 2D angiograms. For example, the collocation points may be determined by: determining minimum and maximum ranges for each of x, y, and t to define a solution space; and sampling the solution to generate a set of random collocation points (e.g., using a technique for random sampling such as, for example, Latin hypercube sampling, Monte Carlo sampling, etc.)

The processor may be further configured to co-register a 3D geometry of the vasculature with the 2D geometry and adjust contrast data at each collocation point based on the co-registered geometries to correct for foreshortening. The processor may be configured to determine flow characteristics (e.g., velocity, pressure) at each collocation point using a physics-informed neural network (PINN) based on the contrast data and collocation points. In some embodiments, the PINN has three inputs (collocation points in x, y, and t). In some embodiments, the PINN has four outputs (C, u, v, and p). In some embodiments, the PINN is a fully connected neural network such that all inputs and all outputs are connected at each layer of the PINN.

The processor may be in communication with and/or include a memory. The memory can be, for example, a random-access memory (RAM) (e.g., a dynamic RAM, a static RAM), a flash memory, a removable memory, and/or so forth. In some instances, instructions associated with performing the operations described herein (e.g., determining flow characteristics, etc.) can be stored within the memory and/or a storage medium (which, in some embodiments, includes a database in which the instructions are stored) and the instructions are executed at the processor.

In some instances, the processor includes one or more modules and/or components. Each module/component executed by the processor can be any combination of hardware-based module/component (e.g., a field-programmable gate array (FPGA), an application specific integrated circuit (ASIC), a digital signal processor (DSP)), software-based module (e.g., a module of computer code stored in the memory and/or in the database, and/or executed at the processor), and/or a combination of hardware- and software-based modules. Each module/component executed by the processor is capable of performing one or more specific functions/operations as described herein. In some instances, the modules/components included and executed in the processor can be, for example, a process, application, virtual machine, and/or some other hardware or software module/component. The processor can be any suitable processor configured to run and/or execute such modules/components. The processor can be any suitable processing device configured to run and/or execute a set of instructions or code. For example, the processor can be a general purpose processor, a central processing unit (CPU), an accelerated processing unit (APU), a field-programmable gate array (FPGA), an application specific integrated circuit (ASIC), a digital signal processor (DSP), and/or the like.

In another aspect, the present disclosure may be embodied as a non-transitory computer-readable medium having stored thereon a program for instructing a processor to perform any of the methods disclosed herein. For example, the program may instruct a processor to obtain time-series angiography data of a vasculature. The program may instruct a processor to determine a vessel wall geometry of the vasculature and generate a set of collocation points in based on the angiography data co-registered with the vessel wall geometry. The program may instruct a processor to determine flow characteristics (one or more of velocity, pressure, and contrast intensity) using a PINN. The PINN may be configured with an objective function based on the Navier-Stokes equation, the convection equation, and boundary conditions including the vessel wall geometry and the angiography data. The objective function may include the continuity equation. In some embodiments, the PINN has three inputs (collocation points in x, y, and t). In some embodiments, the PINN has four inputs (collocation points in x, y, z, and t). In some embodiments, the PINN has four outputs (C, u, v, and p). In some embodiments, the PINN has five outputs (C, u, v, w, and p—where u, v, w correspond to flow velocity in 3D). In some embodiments, the PINN is a fully connected neural network such that all inputs and all outputs are connected at each layer of the PINN. The PINN is configured to derive boundary conditions from the vessel wall geometry and the angiography data.

The program may instruct a processor to determine the flow velocity and pressure at each collocation point by minimizing a difference between a contrast intensity, Cᵣₑₐₗ, measured at each collocation point of the angiography data and an imputed contrast intensity, C_{pred}, calculated from the PINN-determined flow velocity and pressure at each collocation point. Determining the flow velocity and pressure may include generating an angiogram based on the flow velocity and pressure at each collocation point. For example, a 2D angiogram may be generated to aid in controlling convergence of the PINN. In another example, a 3D angiogram is generated to minimize an error in the determined flow velocity and pressure.

For example, the program may instruct a processor to extract a 2D geometry from a time series of 2D angiograms of the vasculature during contrast injection. For example, the 2D geometry may be extracted by averaging two or more of the time series of 2D angiograms to obtain a temporally averaged angiogram; thresholding the temporally averaged angiogram to determine a vessel mask; and filtering the vessel mask with a Sobel filter to obtain a 2D geometry of the vasculature. The program may instruct a processor to determine collocation points (x,y,t) of the time series of 2D angiograms. For example, the collocation points may be determined by: determining minimum and maximum ranges for each of x, y, and t to define a solution space; and sampling the solution to generate a set of random collocation points (e.g., using a technique for random sampling such as, for example, Latin hypercube sampling, Monte Carlo sampling, etc.)

The program may instruct a processor to co-register a 3D geometry of the vasculature with the 2D geometry and adjust contrast data at each collocation point based on the co-registered geometries to correct for foreshortening, the program may instruct a processor to determine flow characteristics (e.g., velocity, pressure) at each collocation point using a physics-informed neural network (PINN) based on the contrast data and collocation points. In some embodiments, the PINN has three inputs (collocation points in x, y, and t). In some embodiments, the PINN has four outputs (C, u, v, and p). In some embodiments, the PINN is a fully connected neural network such that all inputs and all outputs are connected at each layer of the PINN.

### Further Discussion and Test Embodiments

The following are non-limiting example embodiments intended solely to illustrate the present disclosure.

PINNs represent a class of neural networks used to approximate solutions to partial differential equations (PDEs) describing real physical phenomena. These networks leverage several properties of standard feed-forward DNNs to efficiently calculate convergent solutions, including the concepts of automatic differentiation and loss function embedding.

In order to correctly identify the direction in which network weights should be modified to arrive at a more accurate result, DNNs track gradients within the network as part of their training protocol. Within fully connected DNNs, inputs and outputs are connected at each layer of the network, allowing a view of the outputs of the network as a high-ordered function of the inputs, modeled as:

$$Output = NN ( Input ) \qquad ( 12 )$$

where NN represents the set of operations performed upon an input to generate the corresponding output. Since the gradients are tracked at all nodes of the network, partial derivatives of output variables with respect to input variables can be calculated using automatically calculated gradients, preventing the need for additional calculations within the network and improving training times.

With the ability to calculate variables and partial derivatives present in physics-based PDEs, PINNs still need a method of ensuring the equations themselves are satisfied. This is achieved by loss function embedding. Although PDEs typically contain terms on both sides of the equation, algebraic rearrangement of terms to set one side of the equation equal to zero allows the use of the equation as a loss function component. Similarly, any initial conditions (ICs) or boundary conditions (BCs) of the system can be rearranged to become minimization problems. In each iteration of the network, outputs are generated based upon the inputs of the network, partial derivatives are extracted from the gradients present in the network architecture, and all predicted or derived terms are fed into the multicomponent loss function. The net loss tracked by the network is the sum of the squares of each individual loss component. This means that no ground truth labels are required to train a PINN, and that network convergence necessitates that real governing physical equations are respected by network outputs.

### Angiography-Based PINNs

With the characteristics of PINNs in mind, we focus on a network which approximates the solution to the Navier-Stokes equation based upon information from angiographic sequences. The Navier-Stokes equation in one dimension (x-dimension) is:

$$ρ ( \frac{\partial u}{\partial t} + u \frac{\partial u}{\partial x} + v \frac{\partial u}{\partial y} ) = - \frac{\partial p}{\partial x} + μ ( \frac{{\partial }^{2} u}{\partial {x}^{2}} + \frac{{\partial }^{2} u}{\partial {y}^{2}} ) \qquad ( 13 )$$

where (x,y,t) are spatiotemporal coordinates within the vessel, u is the x-component of blood velocity, v is the y-component of blood velocity, p is the pressure in the vessel, ρ is the blood density, and μ is the kinematic viscosity. Using this equation, we can infer our example network should include inputs for 2D spatial coordinates (x,y) as well as a temporal component (t). With these inputs, the network should be able to derive velocities and pressures (u, v, p). This network assumes blood density and viscosity from literature values, as well as an incompressible fluid flow. This network, based strictly on the geometry of the vessel, is modeled by:

$$( u , v , p ) = NN ( x , y , t ) \qquad ( 14 )$$

Much like CFD, this relatively simple implementation of PINNs uses adherence to an inlet velocity function to prevent the network from predicting zero velocity and pressure everywhere within the vessel geometry. This function provides that velocity at the inlet is known (or closely approximated), both across the inlet, and throughout the acquisition. While this can be enforced successfully within a PINN, a beneficial model should not make such assumptions, as patient-specific flow conditions may differ from the approximated model.

The presently-disclosed techniques utilize the contrast propagation throughout an angiographic acquisition as the driving force for non-zero velocity predictions. This is enforced using the convection equation:

$$\frac{\partial C}{\partial t} = - u \frac{\partial C}{\partial x} - v \frac{\partial C}{\partial y} - Pec ( \frac{{\partial }^{2} C}{\partial {x}^{2}} + \frac{{\partial }^{2} C}{\partial {y}^{2}} ) \qquad ( l5 )$$

where (x,y,t) are spatial and temporal coordinates within the vessel, u is the x-component of blood velocity, v is the y-component of blood velocity, C is the contrast density, and Pec is Peclet's number, the ratio of convection to diffusion within the solution space. A network based solely on this equation would have inputs (x,y,t), which would be used to derive terms (u, v, C), assuming Peclet's number is known (or approximately known). Of course, we also have actual propagation of contrast through the vessel, captured throughout the angiographic sequence. As a result, we can add the contrast distribution in (x,y,t) as a boundary condition of the network:

$${C ( x , y , t )}_{pred} = {C ( x , y , t )}_{real} \qquad ( 16 )$$

where C(x,y,t)_{pred} is the approximation of contrast propagation throughout the sequence from the PINN, and C(x,y,t)ᵣₑₐₗ is the actual contrast propagation captured within the angiographic sequence. To summarize, the present PINN will predict contrast density, velocity (in x, y) and pressure fields based upon vessel coordinates in (x,y,t), shown as:

$$( u , v , p , C ) = NN ( x , y , t ) \qquad ( 17 )$$

This network assumes blood density and viscosity, as well as Peclet's number, from literature values, and is further constrained by the boundary condition of real contrast density distributions from the angiographic data.

### Generating Network Inputs

In the test embodiments, the input data to this network was 1000 fps high-speed angiography (HSA) data. HSA data is typically generated in vitro, using patient-specific, 3D printed vascular models derived from CTA in a benchtop flow loop setup. As frames are acquired, a programmable injector introduces iodinated contrast into the vessel, capturing high fidelity contrast flow information. CFD-derived in silico HSA has also been generated, simulating high fidelity contrast flow using the passive scalar method. These simulated HSA acquisitions mitigate concerns over obstruction of flow details due to quantum mottle in in vitro HSA.

The following example utilizes an in vitro HSA acquisition of a carotid artery bifurcation; however, the method was also translated to a passive scalar-generated basilar artery saccular aneurysm sequence and a simple carotid artery bend.

The first step in successful convergence of a PINN was the definition of an accurate solution space having thousands of points located in two Cartesian dimensions, as well as one temporal dimension. First, the HSA input was pre-processed to better isolate contrast signals in each frame, then was masked via mean intensity binary thresholding of the temporally averaged sequence. The walls of the vessel (i.e., vessel wall geometry) were identified via passage of a Sobel filter over the binary mask of the vessel. Each of these steps is shown in FIG. 1.

With the vessel lumen and walls coordinatized by each mask (FIG. 1, middle and FIG. 1, right, respectively), a separate algorithm was tasked with generating collocation points, the coordinatized points input into the network, from these masks. At each row of the mask, this new algorithm identified the bounds of the model as well as the thickness of the vessel lumen (the distance between bounds). Using this information, a random sample of collocation points was generated between the bounds of the row. The number of collocation points generated per row was dependent on the vessel lumen thickness at that row, as well as a user-defined sampling density parameter. This process was repeated for each row in the vessel geometry, creating a complete spatial sampling of the vessel, then the entire sampling process was repeated at each time step of the original acquisition, yielding a random, continuous spatial sampling with discrete temporal sampling. This sampling technique was also performed at the vessel wall to create a subset of points labelled as wall coordinates. In all cases, the known temporal resolution and pixel pitch of the detector were utilized to convert pixels and frames to real physical units in (x,y,t). FIG. 2 shows the collocation points generated for one time step, as well as the points generated across the entire sequence.

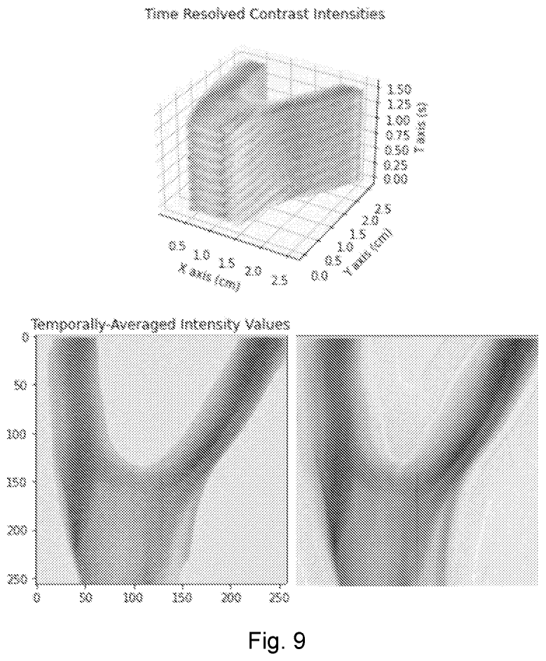

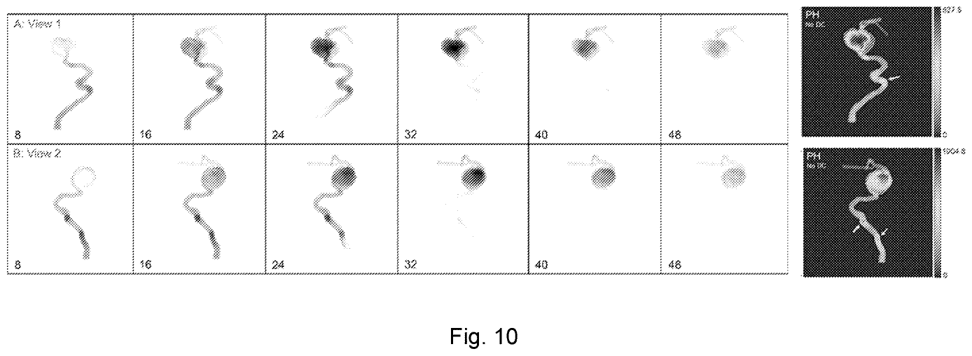

With the described method of the test embodiment, a solution space definition optimized for input into a PINN had been correctly generated solely from angiographic data. This said, the discussed boundary condition of adherence to real contrast propagation from the original HSA sequence could not be enforced with spatial coordinates alone. As a result, a contrast intensity was assigned to each collocation point. This was done by converting the real dimensional coordinates of each collocation point back to pixel and frame coordinates, then assigning the nearest neighbor contrast intensity to the point from the original HSA data. This contrast assignment is shown in FIG. 9. If needed, contrast intensities may also be inverted prior to network input, such that there is a positive correlation between contrast media density and intensity in the image domain.

### Loss Function Encoding

With our inputs defined, the last information required by the network is the governing physical equations themselves. For this network, we enforced the Navier-Stokes equations (Eqs. 3-4), which relates vessel geometry to velocity and pressure gradients, the convection equation (Eq. 5), which relates contrast flow to velocity fields and the conservation of momentum equation (Eq. 6), which can be derived from network outputs and assists the continuity of velocity predictions in the network. Additionally, we enforced a no slip condition at the vessel wall (Eq. 7), and the adherence of the contrast intensity prediction from the network to intensities captured via HSA (Eq. 8).

Of course, even with the manipulation of each equation to create objective functions, neural networks track their loss based upon a singular value, creating a challenge for multicomponent loss functions such as this. Using an assumption that all loss components should be weighted equally, the net loss function for this network is the sum of the squares of each individual loss component (Eqs. 9-10)

### PINN Structure and Hyperparameters

As discussed previously, PINNs are a subset of fully connected DNNs. The PINN utilized in the present tests contained a 3-node input layer (for (x,y,t) coordinates) and 4-node output layer (for (u, v, p, C) outputs) and included 10 hidden layers, each with 250 nodes. The weights at each node were initialized using Xavier initialization and were then adjusted at each epoch of the network. Each collocation input was seen by the network once per epoch, meaning all inputs were used for each training step. The network was trained for 3000 epochs using an Adam optimizer gradient descent method with a learning rate of 1E-3. Individual loss components were tracked separately for plotting purposes, however only the net loss function (Eq. 10) is used for network training.

### Results

Including all pre-processing of image data, collocation point generation, contrast intensity assignment and PINN training, the average minimum convergence time was 23 minutes. This is a marked improvement over CFD velocimetry, in which manual pre-processing of inputs may already extend the analysis past these benchmark times prior to iterative solution generation, which may range from several hours to days, depending on the mesh size, resolution and convergence criteria. Sample results for the in vitro carotid bifurcation acquisition, as well as two in silico HSA acquisitions are shown in FIGS. 5-7. The component velocity and pressure fields have been normalized to their maximums for distribution clarity. FIG. 8 compares contrast predictions for the various models to the corresponding in vitro or in silico HSA image data.

From the results, we observe good qualitative agreement between expected velocity and pressure distributions and PINN outputs. For the in vitro HSA model, we observe that the greatest velocity predictions appear to be in regions with maximal contrast media convection, indicating an adherence to the convection equation, shown in Eqs. 5 and 15. We also observe velocity distributions which reach 0 at the walls of the model, indicating that the network is also closely able to approximate Eq. 7. The pressure component of the PINN prediction for the basilar tip aneurysm shown in FIG. 6 correctly assumes a low pressure in the center of the aneurysm, where the vorticity of blood flowing through the model would create this low-pressure system. We similarly observe the convection equation and no-slip wall condition being respected by this PINN output, as the velocity components follow the convection of contrast around the aneurysm and eventually to the outlets on either side of the model. Finally, in the healthy carotid artery model, we observe a similar trend, in which contrast and velocity measures appear to match expected results, however, the pressure component of the flow appears erroneous, in that the flow appears to move from a low-pressure region to a high-pressure region.

Overall, the results demonstrate the ability of this network to take in raw image sequence inputs, convert the input into a series of points in (x,y,t) with associated contrast intensities, and input these points into a trainable network which is forced to respect underlying governing fluid mechanics equations in its prediction. While many other iterative solvers, including early PINNs and CFD rely on a user-defined inlet velocity function to drive velocity (and subsequently pressure) distributions, the presently-disclosed PINN utilizes contrast media propagation and the convection equation as the driving forces behind velocity distributions. The Navier-Stokes equations are then able to derive pressure fields throughout the model based on these convection equation-derived velocities. It is advantageous for such networks, with no defined inlet velocity function, to properly respect the contrast propagation throughout the image sequence (handled by Eq. 8), as well as the convection equation, as all other terms can easily converge in the case that velocity everywhere is equal to zero. This concern is addressed by the comparison between contrast distributions and real image data shown in FIG. 8. Excluding the background, which falls outside the prediction domain of the PINN and is thus left blank, there is a strong correlation between predicted contrast intensities and HSA contrast intensities. Similarly, as observed in FIG. 7, there is a strong correlation between velocity distributions and the propagation of contrast throughout each model, confirming the hypothesis that inlet velocity functions can be replaced with angiographic image data as the driving force for velocity predictions of iterative solvers. This does also restrict velocity predictions to regions filled with contrast at the given time step, however this issue is partially negated by the Navier-Stokes loss components, as well as the conservation of momentum component (Eq. 6), which enforce continuity and smoothing of velocimetry results. Even so, with pressure currently being an unconstrained variable of the network (which is only constrained by the Navier-Stokes equation-based loss component on the basis of accurate velocity predictions throughout the model), blood velocity distributions distal to the leading edge of contrast media tend to be underapproximated. This limitation of the method also helps explain the error in the pressure distribution shown in FIG. 7.

In the test embodiments, the PINNs were retrained for each input model, rather than loading in and predicting on a pre-trained network. The training time was dependent on the number of collocation points generated, the desired level of acceptable error in the results, and the input image quality. For example, the in vitro HSA carotid bifurcation example took the longest of the three models due to its high temporal resolution, larger number of total frames (1500 frames relative to 650 frames for the next longest acquisition), and injection complexity. Rather than using a single, large bolus, this acquisition used a number of finer contrast media “edges”, which required more training time to properly resolve. This, in addition to the quantum mottle present in the in vitro acquisition (which was not present in either of the two in silico HSA acquisitions) necessitated increased training times to reach a convergent result. Additionally, due to the increased number of frames present in this acquisition, a significantly larger number of collocation points were generated to sample this model, meaning the network was exposed to significantly more inputs per training epoch, leading to slower training times.

It is additionally noteworthy that, although the proposed PINN may need retraining for each HSA acquisition, there are methods by which training times could be expedited. First, an initial velocity distribution could be generated algorithmically (non-iteratively) prior to PINN training and could be used as an initial condition of the network. For example, the contrast dilution gradient (CGD) method, which has shown promise in delivering approximated velocity distributions from raw HSA data, could be used to create an initial, noisy velocity distribution, then PINNs could be used to further refine and denoise these values. Since this represents a relatively simple task for an iterative solver, convergence of this network could take much less time. In addition, some test embodiments of the present disclosure approximated 750 epochs of PINN training providing for consistent convergence, and similar training rates were assumed across all models. This assumption is not intended to be limiting. Other epoch limits may be used. In some tests, PINN convergence was achieved in as few as 350 epochs (or less), with a minimum time of 11 minutes. In some embodiments, an error tolerance threshold could replace this epoch limit as the convergence criteria of the network to further improve its efficiency.

### ADDITIONAL DISCUSSION

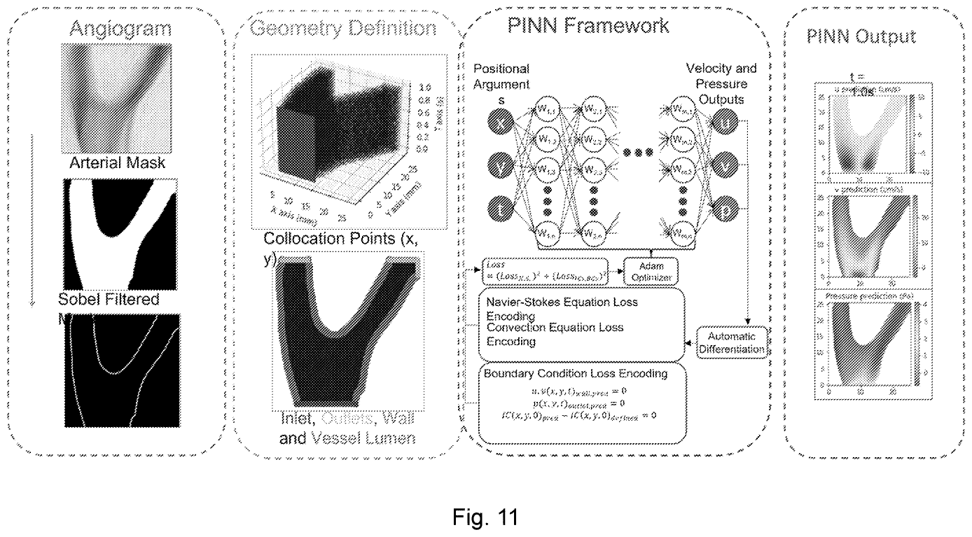

This section presents additional discussion of the example application of PINNs with angiography to derive hemodynamic information in a carotid bifurcation (see FIG. 11). The inputs for the model in this example were the 2D spatial x(x,y) and temporal (t) coordinates, with the network generating the velocity components u(u,v) and pressures p(x,t) respectively. The model assumed constant blood density and viscosity. Based on previous work, an informed selection of the training boundaries in the regions where there were sufficient gradients in the concentration C of the passive scalar, i.e., contrast, eliminated the requirement of imposing velocity and pressure boundary conditions. Consequently, no inlet velocity or pressure functions were employed as initial constraints. In the example model, the propagation of contrast observed in angiography informed the non-zero velocity predictions and followed the convection equation. The equations and boundary conditions were applied as follows:

$${\{ \begin{matrix}ρ ( \frac{\partial u}{\partial t} + ( u \cdot ∇ ) u ) = - ∇ p + μΔ u \\ \frac{\partial C}{\partial t} + ( u \cdot ∇ ) C = 0 \\ ∇ u = 0\end{matrix}}_{\underbrace{Driving Equations}} {\{ \begin{matrix}{u}_{wall} ( x , t ) = 0 \\ {C}_{PINNs} ( x , t ) = {C}_{angio} ( x , t )\end{matrix}}_{\underbrace{Boundary Conditions}} \qquad ( 18 )$$

The driving equations were the 2D Navier Stokes equations, the convection equation where the diffusion term and contrast source were neglected, and the continuation equation. Real x-ray angiographic data was utilized to define the boundary conditions. The boundary conditions were a wall no slip condition and the contrast presence from simulation should be equal with the contrast recorded from the angiographic data. The implemented loss function was written as:

$$Loss = {\underbrace{{\int }_{Ω} {‖ ρ ( \frac{\partial u}{\partial t} + ( u \cdot ∇ ) u ) + ∇ p - μΔ u ‖}^{2} d Ω}}_{Navier - Stokes loss} + {\underbrace{{\int }_{Ω} {‖ \frac{\partial C}{\partial t} + ( u \cdot ∇ ) C ‖}^{2} d Ω}}_{Convection} + {\underbrace{{\int }_{Ω} {‖ ∇ u ‖}^{2} d Ω}}_{Continuity} + {\underbrace{{\int }_{\partial Ω} {‖ u ‖}^{2} d ( \partial Ω )}}_{No - Slip Boundary Condition} + {\underbrace{{\int }_{Ω} {‖ {C}_{PINNs} ( x , t ) - {C}_{angio} ( x , t ) ‖}^{2} d Ω}}_{Contrast pattern match} \qquad ( 19 )$$

Angiographic data was generated using a 3D-printed carotid bifurcation phantom (FIG. 11—Angiogram), which presented an ideal situation with virtual no tortuosity and high level of symmetry as concerns the flow pattern. The phantom was placed parallel with the x-ray imaging detector to minimize shortening.

In refining the contrast match boundary condition (i.e., the second boundary condition in equation (18)), it is noted that angiographic images are a product of x-ray attenuation as described by Beer's Law, I=I₀·exp(−ϑx), where ϑ represents the attenuation coefficient, indicative of the presence of iodinated contrast within the region of interest, and x denotes the material's thickness. To transform this inherently non-linear relationship into a linear one, we first divided the images without contrast (the mask) from those with contrast flow, and subsequently applied a logarithmic operation. This procedure yielded an image that linearly correlates with the product Ox. In this experimental embodiment, we posited a uniform material thickness throughout the flow's projection domain. A grayscale value scale was introduced, ranging from zero (no contrast) to one (complete contrast saturation), to provide that the terms in the contrast-match constraint (equation (18), second boundary condition), correspond to the same attenuation values.

After the contrast adjustment, we established the flow domain through a pre-processing workflow. This involved thresholding the contrast-enhanced images to create a binary mask of the vessel lumen (FIG. 11—Arterial Mask), and employing a Sobel filter to outline the vessel boundaries, thus defining the no-slip condition (FIG. 11—Sobel Filter). Spatial and temporal collocation points were then systematically sampled within the vessel lumen, using a predetermined sampling density parameter (FIG. 11—Geometry Definition). The known temporal resolution and pixel size of the x-ray detector facilitated the conversion of image pixels and time frames into physical coordinates (x,y,t).

Following this, we applied the PINNs for the two-dimensional flow modeling, guided by the loss function specified in equation (19). The boundary conditions for inlet velocity and pressure were considered unknown. The network architecture incorporated an input layer with three nodes, an output layer with four nodes, and ten hidden layers. Training was conducted using the Adam optimizer, a learning rate of 1E-3 (0.001), over 3000 epochs. The PINNs displayed consistent convergence, with an average minimum convergence duration of 23 minutes, under steady flow conditions. Although the velocity profiles qualitatively matched expectations (FIG. 11—PINNs Output), there were discrepancies up to 50% in magnitude compared to bulk flow measurements from a flow sensor mounted on the inlet.

The above discussion validated the use of real-world angiographic data for hemodynamic modeling and that PINNs can infer hemodynamics with incomplete boundary information using image-based constraints. By enhancing the precision of the contrast match loss, more accurate results can be achieved. In some cases, two-dimensional PINNs models are insufficient for detailed hemodynamic studies from standard projection angiography. Instead, a three-dimensional approach may be used.

### PINNs Application to Extract Hemodynamic Information in Neurovascular Phantoms.

The objective of this section is to formulate, evaluate, and validate a PINNs-centric approach that leverages angiographic data to infer local hemodynamics in neurovascular diseases. Incorporating angiographic data as auxiliary constraints can effectively compensate for missing or unknown boundary conditions, thereby facilitating the PINNs's convergence to a more accurate hemodynamic solution. Building upon the established relationship ability of the PINNs to model three-dimensional vascular flow dynamics, this work provides an in-depth understanding of key algorithmic considerations. Specifically, we provide configurations for loss functions, activation functions, density of collocation points, and the tolerable extent of neglected boundary conditions. Methodologically, we describe various computational methodologies to include image constraints in the PINNs algorithm. We describe a system capable of providing precise hemodynamic measurements alongside high-resolution temporal and spatial angiographic data, using patient-specific phantoms representing two distinct types of neurovascular disease, i.e., intracranial aneurysms and intracranial atherosclerotic disease (ICAD).

Improve Image Matching Term in PINN Loss Function to Account for X-Ray Interaction with the Contrast and Patient Vascular Geometry.

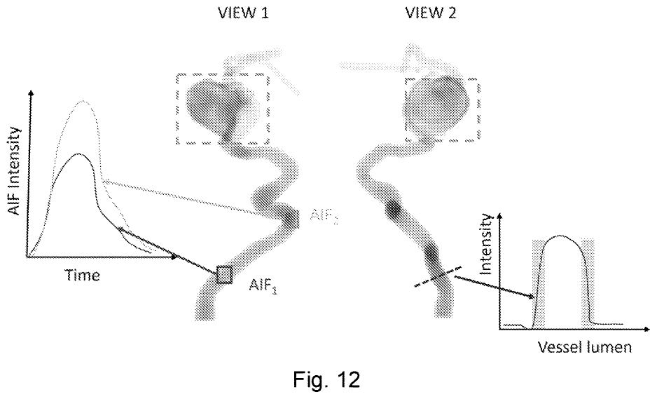

The use of two-dimensional (2D) angiographic images, even when biplane imaging is employed, can have limitations when integrated into PINNs with image-based constraints. This is due to the fundamental principles of X-ray image formation, where the intensity at each pixel depends on the integrated distribution of the imaged substance and the attenuation of X-rays, as described by Beer's law. In digital subtraction angiography (DSA), a mask image is typically acquired prior to contrast injection and subsequently subtracted from post-contrast images to display only the vasculature filled with contrast media. However, while this imaging processing step may be used, it is not sufficient to provide a reliable imaging tool for hemodynamics related parameters extraction due to the challenges posed by the intricate angioarchitecture inherent in neurovascular systems. FIG. 12 depicts an internal carotid intracranial aneurysm and illustrates the complexities inherent in DSA imaging and the ensuing challenges in discerning contrast gradients and local iodine concentrations.

The utilization of PINNs in this context is further complicated by the need to employ the convection equation, which utilizes temporal derivatives and spatial gradients of contrast to characterize the media flow within a dynamic fluid system. In contrast to previous work, some embodiments of the present disclosure involve a modified convection equation that omits the diffusion term (because convection is the dominant phenomena in angiography), but retains the source term at the point of contrast injection. Inclusion of the contrast injection profile is advantageous as the injection parameters—such as contrast volume, mixing, duration, and velocity—can dramatically influence contrast propagation.

To clarify the intricacies of X-ray projection imaging and its associated potential errors in the PINNs methodology, consider the following (with reference to FIG. 12, involving contrast injection through a primary artery, such as the carotid, and subsequent 2D quantitative angiography performed on an aneurysm).

Positioning a region of interest (ROI) on the primary artery captures the contrast injection profile to compensate for variability. However, shifts in ROI placement along the artery could introduce errors due to foreshortening, leading to intensity measurements that are dependent on the location of the ROI. It can be, therefore, advantageous to define a contrast injection function that is consistent regardless of the measurement point at the artery's inlet.

When employing a biplane imaging system to measure the ROI at the aneurysm's dome, the varying path lengths traversed by the X-rays could produce disparate intensity readings. This variability underscores the benefits for additional constraints that consider the three-dimensional path of X-rays through the aneurysm dome, suggesting that bi-plane X-ray views might generate inconsistent constraint images for the PINNs.

Lastly, the application of the convection equation to estimate contrast gradients posits that the product of the spatial contrast gradient and local velocity equates to the time derivative at any point within the artery. However, a line profile across the primary artery would typically exhibit a parabolic pattern, indicating erroneously high velocities perpendicular to the vessel wall—this is an artifact arising from decreased path length near the vessel's periphery. A path length correction could theoretically rectify this, assuming the edge singularities of the artery are accounted for and adjusted.

These examples reinforce the notion that the incorporation of precise 2D constraints into PINNs may involve supplementary three-dimensional (3D) data to substantively enhance the diagnostic capabilities of intraoperative angiograms. Some embodiments of the present disclosure provide methodologies that can be integrated into the PINNs framework, including the incorporation of 3D geometry and imaging data, to overcome these challenges. To account for patient geometry and the process of planar image formation, some embodiments of the present disclosure modify the image-driven constraint in the PINNs algorithm. Computational burdens can be balanced with the practicability of the system as desired for particular applications. As a first step, the hemodynamic equations may be solved in 3D space:

$${\underbrace{\{ \begin{matrix}ρ ( \frac{\partial u}{\partial t} + ( u \cdot ∇ ) u ) = - ∇ p + μΔ u \\ \frac{\partial C}{\partial t} + ( u \cdot ∇ ) C = S ( x , t ) \cdot δ ( x - {x}_{inl} ) \\ ∇ u = 0\end{matrix}}}_{Driving Equations} \qquad ( 20 )$$

$$\{ {\underbrace{\begin{matrix}{u}_{wall} ( x , t ) = 0 \\ Image constraint\end{matrix}}}_{Boundary Conditions}$$

These are like equation 18, except that now we use 3D vectors, and the contrast injection term has been introduced for the inlet region. For the image constraints, the incorporation of image information can be performed in two ways. The first approach is to solve the flow equations (20) and the associated loss function, then project the contrast flow solution from the PINNs onto the image planes. The discrepancy between this projection and actual DSA image data will be minimized, represented by the following boundary condition:

$$Image constraint : \{ \begin{matrix}{Proj}_{1} ( {C}_{PINNs} ( x , t ) ) = {I}_{1} ( t ) \\ {Proj}_{2} ( {C}_{PINNs} ( x , t ) ) = {I}_{2} ( t )\end{matrix} \qquad ( 21 )$$

where Proj₁(C_{PINNs}(x, t)) and Proj₂(C_{PINNs}(x, t)) are the cone beam forward projection operators mapping the concentration distribution C_{PINNs}(x, t) from the PINNs to the detector planes, and I₁(t) and I₂(t) are the logarithmically corrected DSA images at time t.

The second approach, which is computationally more involved, is to reconstruct the flow of contrast from sparse data (e.g., from two views) and minimize the difference between the reconstructed 3D image and the PINNs convection solution. In this condition, the image constrain may be written as follows:

$$Image constraint : {C}_{PINNs} ( x , t ) = {C}_{recon} ( x , t ) \qquad ( 22 )$$

$$with {C}_{recon} ( x , t ) = {ℛ}^{- 1} ( {I}_{1} ( t ) , {I}_{2} ( t ) )$$

where [symbol] ⁻¹ is the reconstruction operator based on I₁(t) and I₂(t) information.

Within the framework of these methodologies, an advantageous step involves the co-registration of image data with the predictions generated by the PINNs. This process will be divided into two distinct methodologies which will be applied separately to the two approaches described in the paragraphs above. The first approach utilizes a 3D-to-2D coregistration, where the orientation of the 3D PINNs contrast flow solution which coincides with the 2D x-ray imaging planes is determined. The second approach utilizes a 3D-to-3D coregistration, which aligns the PINNs contrast flow output directly with volumetric 3D reconstructed imaging data of the flow of contrast agent.

Once the co-registration parameters have been determined, there is no need to recalibrate the transformation parameters for subsequent analyses. This streamlines the process, allowing for swift and reliable integration of data sets. The Astra toolbox and SimpleElastix libraries within Python were leveraged to execute this task.

For the 2D-to-3D PINNs co-registration, the contrast flow deduced from the PINNs model is accurately aligned with the projection data obtained from the x-ray detectors. An example methodology we have devised is a two-phase registration protocol. Initially, the 3D structure as defined within the PINNs framework is projected onto two distinct x-ray imaging planes, which may be denominated as the lateral and frontal views, in alignment with the conventions employed in contemporary neuro-interventional suites. Subsequently, utilizing SimpleElastix, an affine co-registration is conducted, harmonizing the PINNs projections with the x-ray images, and we quantify the alignment using the Dice similarity coefficient between the projected data and the true vascular morphology. This calibration process is iteratively refined across a spectrum of angles until an optimal Dice coefficient is achieved. The derived 3D rotation and affine parameters may be recorded for all subsequent PINNs computation in a given model.

The neurovascular arteries' inherent tortuosity and the distinctive anatomy of the Circle of Willis provide a naturally occurring, immutable point of reference-akin to a ‘biological GPS’. It is this unique geometry that ensures only one specific perspective will yield the highest Dice coefficient, reinforcing the reliability of our co-registration method. (The Dice coefficient is often used to compare the similarity of two images, such as a medical image and a segmentation of that image. It can also be used to compare the similarity of two sets of data in general, such as the two approaches for incorporating image information into PINNs that we described herein).

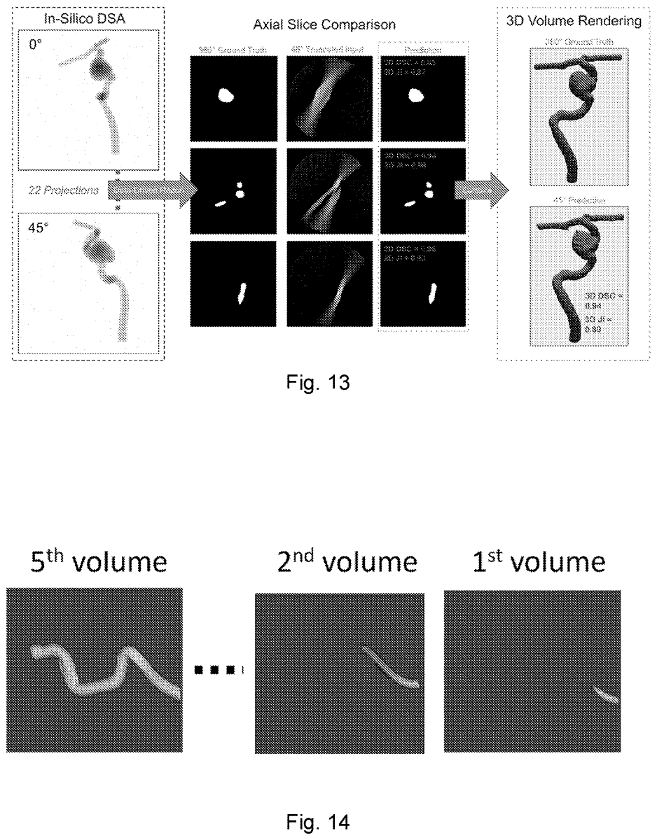

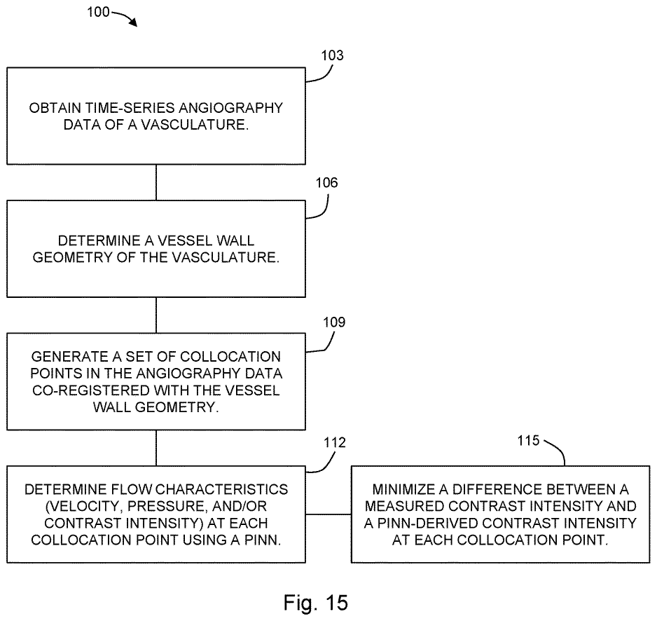

The second approach utilizes 3D-to-3D affine co-registration between the PINNs flow 3D domain and the actual arterial 3D flow domain which is a textbook operation. The challenging task is 3D capture of the contrast flow, i.e., reconstructing a dynamic phenomenon from limited views. This is a reconstruction task that could be computationally more expensive. An example solution is an AI-driven 3D reconstruction method based on Deep Convolutional Generative Adversarial Networks (DCGANs). The key aspect of this process is to acquire rotational data during the passage of the bolus through the region of interest in a few seconds, i.e., during arterial bolus transition. This acquisition is possible with current technology, but one constraint of 3D reconstructions is that the object cannot change significantly from view to view. We can overcome this constraint by using a method that reconstructs the data with a limited number of projections (e.g., 45-60 degrees). We have developed a data-driven method to reconstruct 3D volumes using truncated projection acquisition, where “truncated” refers to 45 projections acquired 1° apart. We tested the DCGAN on simulated and un-simulated 3D angiograms (FIG. 13 and FIG. 14 respectively). We assessed the method's performance by comparing it to the ground truth (standard reconstruction) using various metrics, including line profiles, the modulation transfer function, the noise power spectrum, Hounsfield unit linearity analysis, and Dice coefficients.

To recover contrast flow 3D data, the imaged phantoms may be on a rotational stage which will allow between 3-5 3D reconstructions per second, using only 10 projections per detector. In addition, in an extreme case, two views 3D reconstruction could in theory provide 30 3D volumes per second reconstructions. Of note images in FIG. 14 were generated using two-views reconstruction without a priori knowledge of the 3D reconstruction domain. This data may be used in the loss function to minimize the image difference term.

### Angiographic Image Theory

### Concepts

We understand that contrast density is inversely proportional to image intensity, however, this relationship is not directly quantified. To establish this relationship, we will need to use a modified version of the Beer Lambert equation as it relates to x-ray image formation. The original equation, which factors beam current, beam energy, the linear attenuation coefficient of attenuating materials along the path length, and the path length itself, is shown below:

$$I = {I}_{0} {e}^{- ( μ x )}$$

Where I is the final x-ray intensity. I₀ is the initial x-ray intensity, p is the linear attenuation coefficient, and x is the material thickness (the path length through the material). Beam current is factored into the initial x-ray intensity, I₀, and beam energy is factored into the linear attenuation coefficient, which is energy-dependent. In the case that the beam passes through multiple materials prior to entering the detector plane, the effects are exponentially summative, such that:

$$I = {I}_{0} {e}^{- ( {μ}_{1} {x}_{1} + {μ}_{2} {x}_{2} )}$$

This equation becomes useful for determination of final x-ray intensities after passing through vessels during an angiographic sequence as the vessel composition can be loosely defined as two materials: blood and contrast. The complication, in this case, is that our attenuation coefficients are not static, and are dependent on the fractional concentrations of blood and contrast present in each position in the vessel. The path length over which x-rays are attenuated is also location-specific, requiring some understanding of the 3D structure of the vessel. We will use these angiography concepts in addition to the multicomponent Beer Lambert equation above to derive a relationship between contrast density and x-ray attenuation. In the case that a photon-counting detector is used for image formation, we also know that final x-ray intensity is directly proportional to pixel intensity in the generated image, whereas indirect detectors may require a conversion factor relating to the incident beam energy.

### Derivations

### Variable Definitions:

Let x and y be the in-plane spatial coordinates of the vessel.

Let z(x,y) be the depth component of the vessel. We will abbreviate this to z.

Let c(x, y, z, t) be the relative contrast density in a given voxel such that 0≤c(x, y, z, t)≤1.

Let C(x,y,t) be the average contrast density in a given pixel, such that:

$$C ( x , y , t ) = \frac{\int c ( x , y , z , t ) dz}{z}$$

We then know 0≤C(x,y)≤1.

Let μ_{c} and μ_{B} be the linear attenuation coefficients of contrast and blood, respectively.

### Equations:

Since we know the relative concentration of contrast in a voxel is c(x, y, z, t), we can also define the relative concentration of blood in that voxel in terms of this concentration, simply as:

$$( 1 - c ( x , y , z , t ) )$$

It then follows that the average blood density in a given pixel would be:

$$( 1 - \frac{\int c ( x , y , z , t ) dz}{z} ) = ( 1 - C ( x , y , t ) )$$

Using these relations and the general form of the Beer Lambert equation, we obtain the following equation:

$$I = {I}_{0} {e}^{- ( C ( x , y , t ) {μ}_{c} + ( 1 - C ( x , y , t ) {μ}_{B} ) z}$$

In the case that the 4D distribution of contrast densities is already known (such as in the raw data from CFD simulations), this equation, and LUT values for μ_{c} and μ_{B}, can be used to obtain the relative relationship between initial and final x-ray intensities by back-converting to 4D coordinates such that:

$$I = {I}_{0} {e}^{- ( ( \frac{\int c ( x , y , z , t ) dz}{z} ) {μ}_{c} + ( 1 - ( \frac{\int c ( x , y , z , t ) dz}{z} ) {μ}_{B} ) ) z}$$

If we assume I₀=1, we can determine a relative relationship between final and initial x-ray intensities for each pixel in (x,y,t) throughout the acquisition, which can then be used along with a scaling factor to generate realistic pixel values for simulated angiography acquisitions.

In the case that we are performing the reverse calculation, we utilize a subtraction image. The equation for the subtraction image is as follows:

$${I}_{s u b} = {I}_{0} {e}^{- ( {μ}_{B} ) z}$$

If we log subtract this image sequence from the angiographic data, we obtain:

$$\frac{I}{{I}_{s u b}} = \frac{{I}_{0} {e}^{- ( C ( x , y , t ) {μ}_{c} + ( 1 - C ( x , y , t ) {μ}_{B} ) z}}{{I}_{0} {e}^{- ( {μ}_{B} ) z}} = {e}^{- ( C ( x , y , t ) {μ}_{c} - C ( x , y , t ) {μ}_{B} ) z}$$

Taking the natural log of both sides, and isolating the contrast density term:

$$ln ( \frac{I}{{I}_{s u b}} ) = - C ( x , y , t ) ( {μ}_{C} + {μ}_{B} ) z$$

$$C ( x , y , t ) z = \frac{ln ( \frac{I}{{I}_{s u b}} )}{( {μ}_{C} + {μ}_{B} )}$$

Using the voxelized definition of the average contrast density, C(x,y,t):

$$C ( x , y , t ) z = \frac{\int c ( x , y , z , t ) d z}{z} z = \int c ( x , y , z , t ) dz = \frac{ln ( \frac{I}{{I}_{s u b}} )}{( {μ}_{C} + {μ}_{B} )}$$

Although this does not solve the contrast intensity in each voxel directly, it gives a method of comparing 4D contrast distributions to actual image data, lending utility to 4D flow algorithms such as PINNs or epipolar reconstructions of biplane angiography.

### Limitations

While these relations hold true for all simulated data, there are several additional considerations that would allow more accurate modeling of these relationships in real imaging applications. For example, we do not consider the effects of anatomical noise. In an embodiment, the log subtraction used to isolate the contrast density term may also factor anatomical noise, however, misregistration of the image sequences could impact the accuracy of this relationship (in any case, the relation determining the attenuation from CFD data does not factor other anatomical structures). The derived relations also assume several characteristics of the acquisition. First, we assume a monochromatic beam, which allows the linear attenuation coefficients to both be known and to be held constant. Next, we assume no vessel motion (this includes both translation motion and dilation of the vessel itself). We also assume 3D vessel structure is known. This is reasonable, as many patients have rotational angiography scans or CTA which can be registered to the planar data, however this also requires proper registration of the 3D model to the projection through the vessel. In the absence of 3D data, a cylindrical vessel model could be used for simple vessels. Finally, we are also not considering quantum mottle in our calculations, however, for the sake of image formation, this could be applied after the final vs initial x-ray relationship is established.

Although the present disclosure has been described with respect to one or more particular embodiments, it will be understood that other embodiments of the present disclosure may be made without departing from the spirit and scope of the present disclosure.

What is claimed is:

1. A computer-implemented method for predicting flow characteristics in a vasculature, comprising:
obtaining time-series angiography data of a vasculature;
determining vessel wall geometry within the vasculature;
generating, using one or more processors, a set of collocation points within the time-series angiography data co-registered with the vessel wall geometry; and
determining, using the one or more processors, a flow velocity and pressure at each collocation point using a physics-informed neural network (PINN) having an objective function based on the Navier-Stokes equation, the convection equation, and boundary conditions including the vessel wall geometry and the angiography data.

2. The computer-implemented method of claim 1, wherein determining the flow velocity and pressure at each collocation point comprises minimizing a difference between a contrast intensity measured at each collocation point of the angiography data and an imputed contrast intensity calculated from the PINN-determined flow velocity and pressure at each collocation point.

3. The computer-implemented method of claim 2, further comprising correcting for foreshortening error in the measured contrast intensity at each collocation point of the angiography data.

4. The computer-implemented method of claim 1, further comprising generating an angiogram based on the determined flow velocity and pressure at each collocation point.

5. The computer-implemented method of claim 4, wherein the generated angiogram is a 2D angiogram to aid in controlling convergence of the PINN.

6. The computer-implemented method of claim 4, wherein the generated angiogram is a 3D angiogram to minimize an error in determined flow velocity and pressure.

7. The computer-implemented method of claim 1, wherein the angiography data comprises a time series of 2D angiograms of the vasculature during contrast injection.

8. The computer-implemented method of claim 1, further comprising obtaining a digital 3D model of the vasculature.

9. The computer-implemented method of claim 1, wherein the angiography data is obtained from magnetic resonance imaging (MRI), computed tomography (CT), or X-ray imaging.

10. The computer-implemented method of claim 1, further comprising determining, using the one or more processors, a state of occlusion of one or more vessels of the vasculature.

11. The computer-implemented method of claim 1, wherein the collocation points of the set of collocation points are randomly selected within the vasculature.

12. The computer-implemented method of claim 1, wherein the PINN has three inputs corresponding to x, y, and t coordinates of the set of collocation points.

13. The computer-implemented method of claim 12, wherein the PINN has a fourth input corresponding to the z coordinate of the set of collocation points.

14. The computer-implemented method of claim 1, wherein the PINN has four outputs corresponding to contrast intensity (C), flow velocity (u, v), and pressure (p).

15. The computer-implemented method of claim 1, wherein the PINN is a fully connected neural network such that all inputs and all outputs are connected at each layer of the PINN.

16. The computer-implemented method of claim 1, wherein the PINN is configured to derive boundary conditions from the vessel wall geometry and the angiography data.

17. A system for predicting flow characteristics in a vasculature, comprising:
a processor, wherein the processor is programmed to:
receive time-series angiography data of a vasculature;
determine vessel wall geometry within the vasculature;
generate a set of collocation points within the time series angiography data co-registered with the vessel wall geometry; and
determine a flow velocity and pressure at each collocation point using a physics-informed neural network (PINN) having an objective function based on the Navier-Stokes equation, the convection equation, and boundary conditions including the vessel wall geometry and the angiography data.

18. The system of claim 17, wherein the processor is programmed to determine the flow velocity and pressure at each collocation point comprises minimizing a difference between a contrast intensity measured at each collocation point of the angiography data and an imputed contrast intensity calculated from the PINN-determined flow velocity and pressure at each collocation point.

19. The system of claim 17, wherein the processor is configured to correct for foreshortening error in the measured contrast intensity at each collocation point of the angiography data.

20. The system of claim 17, wherein the processor is further configured to generate an angiogram based on the determined flow velocity and pressure at each collocation point.

21. The system of claim 20, wherein the generated angiogram is a 2D angiogram to aid in controlling convergence of the PINN.

22. The system of claim 20, wherein the generated angiogram is a 3D angiogram to minimize an error in determined flow velocity and pressure.

23. The system of claim 17, wherein the angiography data comprises a time series of 2D angiograms of the vasculature during contrast injection.

24. The system of claim 17, wherein the processor is further configured to obtain a 3D model of the vasculature.

25. The system of claim 17, wherein the angiography data is obtained from magnetic resonance imaging (MRI), computed tomography (CT), or X-ray imaging.

26. The system of claim 17, wherein the processor is further configured to determine a state of occlusion of one or more vessels of the vasculature.

27. The system of claim 17, wherein collocation points of the set of collocation points are randomly selected within the vasculature.

28. The system of claim 17, wherein the PINN has three inputs (collocation points in x, y, and t).

29. The system of claim 28, wherein the PINN has four outputs (C, u, v, and p).

30. The system of claim 17, wherein the PINN has four inputs (collocation points in x, y, z, and t).

31. The system of claim 30, wherein the PINN has five outputs (C, u, v, w, and p).

32. The system of claim 17, wherein the PINN is a fully connected neural network such that all inputs and all outputs are connected at each layer of the PINN.

33. A non-transitory computer-readable medium having stored thereon a program for instructing a processor to:
obtain time-series angiography data of a vasculature;
determine vessel wall geometry within the vasculature;
generate, using one or more processors, a set of collocation points within the time-series angiography data co-registered with the vessel wall geometry;
determine, using the one or more processors, a flow velocity and pressure at each collocation point using a physics-informed neural network (PINN) having an objective function based on the Navier-Stokes equation, the convection equation, and boundary conditions including the vessel wall geometry and the angiography data.

34. The non-transitory computer-readable medium of claim 33, further comprising instructions to determine the flow velocity and pressure at each collocation point by minimizing a difference between a contrast intensity measured at each collocation point of the angiography data and an imputed contrast intensity calculated from the PINN-determined flow velocity and pressure at each collocation point.

35. The non-transitory computer-readable medium of claim 33, further comprising instructions to correct for foreshortening error in the measured contrast intensity at each collocation point of the angiography data.

36. The non-transitory computer-readable medium of claim 33, further comprising instructions to generate an angiogram based on the determined flow velocity and pressure at each collocation point.

37. The non-transitory computer-readable medium of claim 36, wherein the generated angiogram is a 2D angiogram to aid in controlling convergence of the PINN.

38. The non-transitory computer-readable medium of claim 36, wherein the generated angiogram is a 3D angiogram to minimize an error in determined flow velocity and pressure.
